(19) United States
(12) Patent Application Publication
(10) Pub. No.: US 2013/0079589 A1
(43) Pub. Date: Mar. 28, 2013

(54) INCONTINENCE TREATMENT DEVICE CONFIGURED FOR URETHRAL PLACEMENT INTO THE BLADDER

(71) Applicants: Sarah J. Deitch, (US); Janell Colley, Minneapolis, MN (US); Julie M. Kerkvliet, Eik River, MN (US); Gregg Ledin, Aitkin, MN (US)

(72) Inventors: Sarah J. Deitch, (US); Janell Colley, Minneapolis, MN (US); Julie M. Kerkvliet, Eik River, MN (US); Gregg Ledin, Aitkin, MN (US)

(73) Assignee: COLOPLAST A/S, Humlebaek (DK)

(21) Appl. No.: 13/447,292

(22) Filed: Apr. 16, 2012

Related U.S. Application Data
(60) Provisional application No. 61/538,129, filed on Sep. 23, 2011.

(30) Foreign Application Priority Data
Sep. 22, 2011 (DK) PA 2011 70523

(51) Int. Cl. A61F 2/00

(52) U.S. Cl. 600/32

(57) ABSTRACT

An incontinence treatment device has a solid rod connected between a proximal portion and a distal portion. The proximal portion is insertable into a urinary bladder. The solid rod is configured for placement in the urethra. The solid rod has a length that adapts the distal portion to be positioned outside and distal to the urethra with the proximal portion positioned in the urinary bladder. The proximal portion has a lateral dimension that is at least a factor of 3 greater than a lateral dimension of the solid rod and is so configured to block a neck of the urinary bladder and impede flow of urine out of the urinary bladder. A force applied to the distal portion displaces the proximal portion away from the neck of the urinary bladder to allow urine to exit the urinary bladder.

### BACKGROUND

Devices for treating urinary incontinence include slings, supports, and other scaffold-like devices that are implanted in a patient's body to support the urethra.

A sub-urethral sling is a urinary incontinence treatment device that is surgically implanted under the urethra to support the urethra and inhibit urine from leaking out of the urethra during a provocative event such as coughing or sneezing. Such an incontinence treatment device is typically implanted through one or more incisions and is anatomically secured to supporting tissue(s).

Other urinary incontinence treatment devices, such as injected bulking liquids, are applied to coaptate the urethra. Injected bulking agents have proven effective. However, most injected bulking agents are associated with reduced efficacy over time, the solution of which is to re-inject more bulking agent.

Improved incontinence treatment methods and devices would be welcomed by both the patient and the surgical staff.

### SUMMARY

One aspect provides an incontinence treatment device having a solid rod connected between a proximal portion and a distal portion. The proximal portion is insertable into a urinary bladder. The solid rod is configured for placement in the urethra. The solid rod has a length that adapts the distal portion to be positioned outside and distal to the urethra with the proximal portion positioned in the urinary bladder. The proximal portion has a lateral dimension that is at least a factor of 3 greater than a lateral dimension of the solid rod and is so configured to block an exit of the urinary bladder and impede flow of urine out of the urinary bladder. A force applied to the distal portion displaces the proximal portion away from the exit of the urinary bladder to allow urine to exit the urinary bladder.

One aspect provides an incontinence treatment device including an inflatable proximal portion that is insertable into a urinary bladder, a pump, and a tube connected between the proximal portion and the pump. The pump is positionable distal to an exit of the urethra. The pump is operable to inflate the proximal portion to an expanded dimension that blocks an exit of the urinary bladder to impede flow of urine out of the urinary bladder. The pump is operable to deflate the proximal portion to a contracted dimension that allows urine to flow from the urinary bladder through the urethra.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1A is a perspective view and FIG. 1B is a cross-sectional view of one embodiment of an incontinence treatment system including an incontinence treatment device and an insertion tool.

FIG. 2 is a side cross-sectional view of the device illustrated in FIG. 1.

FIG. 3 is a top view of the device illustrated in FIG. 1.

FIGS. 4A-4C are schematic views of the system illustrated in FIG. 1 being placed in a female.

FIG. 5A is a schematic view of the device illustrated in FIG. 1 blocking a neck of a urinary bladder to impede flow of urine out of the urinary bladder.

FIG. 5B is a schematic view of the device illustrated in FIG. 1 adjusted to position a proximal portion away from the neck of the urinary bladder to allow urine to exit the urinary bladder.

FIG. 6 is a perspective view of one embodiment of an incontinence treatment device.

FIG. 7A is a side view of one embodiment of an incontinence treatment device including a proximal portion attached to a rod at a pivot point.

FIGS. 7B is a side view and FIG. 7C is a front view of the device illustrated in FIG. 7A with the proximal portion rotated into a substantial longitudinal alignment with the rod.

FIG. 8 is a side cross-sectional view of one embodiment of an incontinence treatment device including an inflatable proximal portion in a deflated state.

FIG. 9 is a side cross-sectional view of the incontinence treatment device illustrated in FIG. 8 with the inflatable proximal portion in an inflated state. FIG. 10 is a schematic view of the device illustrated in FIG. 9 when implanted in a female.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

Tissue includes soft tissue, which includes dermal tissue, sub-dermal tissue, ligaments, tendons, or membranes. As employed in this specification, the term “tissue” does not include bone.

The urethra is normally supported by connective and other tissues. Over time, and particularly with parous women, the support of the urethra erodes, which can give rise to hyper-mobility of the urethra. Hyper-mobile urethras are susceptible to the undesirable leaking of urine during provocative events such as sneezing, laughing, or coughing (the persistence of which is sometimes referred to as stress urinary incontinence).

Embodiments provide an incontinence treatment device configured for urethral placement into the bladder. The device includes a proximal portion that is inserted into the urinary bladder and a distal portion that is accessible by the user. The proximal portion is configured to block the exit of the urinary bladder to impede the flow of urine out of the bladder. The distal portion is operable to displace the proximal portion and allow urine to flow out of the urinary bladder. The device is initially placed through the urethra into the bladder, preferably by a surgeon, a gynecologist, or a urologist, without creating an incision. In one embodiment the device is removable by the user (for example for cleaning) and replaceable by the user.

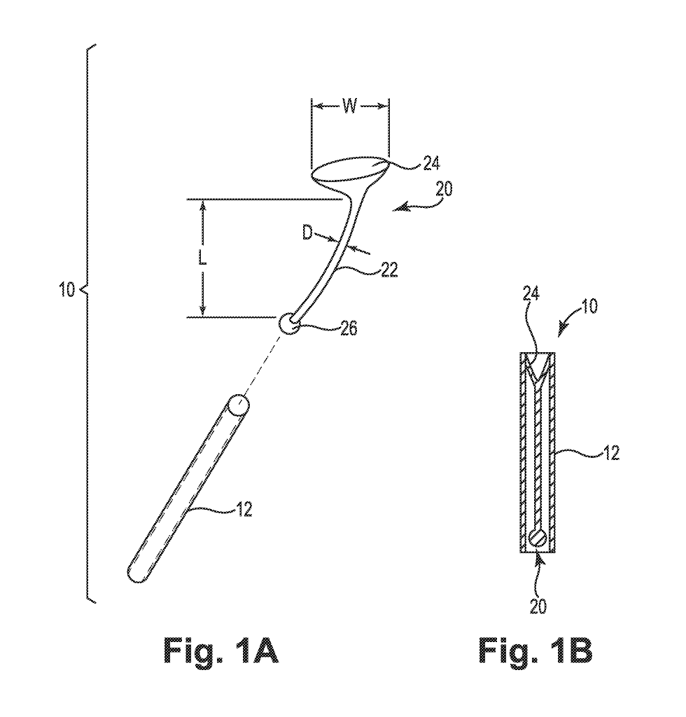

FIG. 1A is a perspective view and FIG. 1B is a cross-sectional view of one embodiment of an incontinence treatment system 10 including an insertion tool 12 and a device 20. The insertion tool 12 is sized for placement into the urethra, and in one embodiment is a rigid tubular cylinder provided with an interior annular space sized to retain the device 20. The device 20 is flexible and compressible to fit inside of the insertion tool 12. The insertion tool 12 is operable to deliver the device 20 through the urethra into the urinary bladder. The insertion tool 12 is removed from the urethra after the device 20 is ejected or pushed out of the insertion tool 12 and placed in the urethra and partway into the bladder of the user.

The device 20 includes a rod 22 connected between a proximal portion 24 and a distal portion 26. The rod 22 is provided with a length L that adapts the distal portion 26 to be positioned outside and distal to the urethra when the proximal portion 24 is positioned in the urinary bladder. The proximal portion 24 is insertable into the urinary bladder and has a lateral dimension W that is at least a factor of 3 greater than a lateral dimension D of the rod 22 and is so configured to block a neck of the urinary bladder to impede flow of urine out of the urinary bladder.

In one embodiment, the length L of the rod 22 is between 2-10 cm, with one acceptable length L of the rod 22 being about 3 cm. In one embodiment, the lateral dimension D of the rod 22 is between 0.5-6 mm, with one acceptable lateral dimension D of the rod 22 being about 1 mm. In one embodiment, the lateral dimension W of the proximal portion 24 is at least a factor of 3 times the lateral dimension D of the rod 22 and has a width ranging from about 1.5-20 mm.

The device 20 is sized according to user anatomy. In one embodiment, the device 20 is provided in sizes of small, medium, and large. The small device 20, in one example, is provided with a length L of the rod 22 of about 3 cm, a lateral dimension D of the rod 22 of about 1.5 mm, and a lateral dimension W of the proximal portion 24 of about 10 mm. The large device 20, in one example, is provided with a length L of the rod 22 of about 5 cm, a lateral dimension D of the rod 22 of about 4 mm, and a lateral dimension W of the proximal portion 24 of about 16 mm.

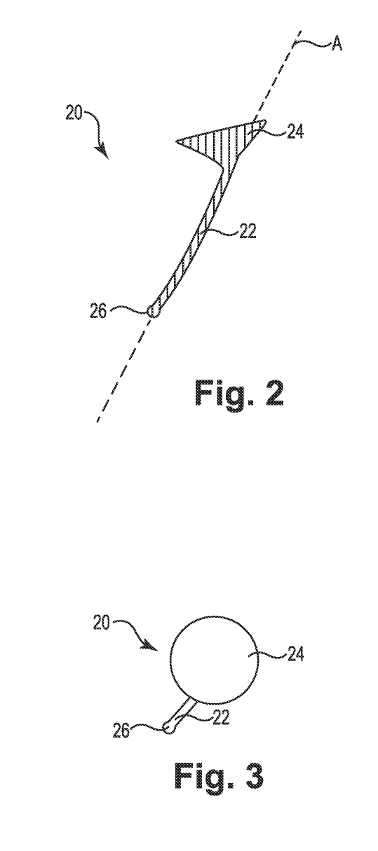

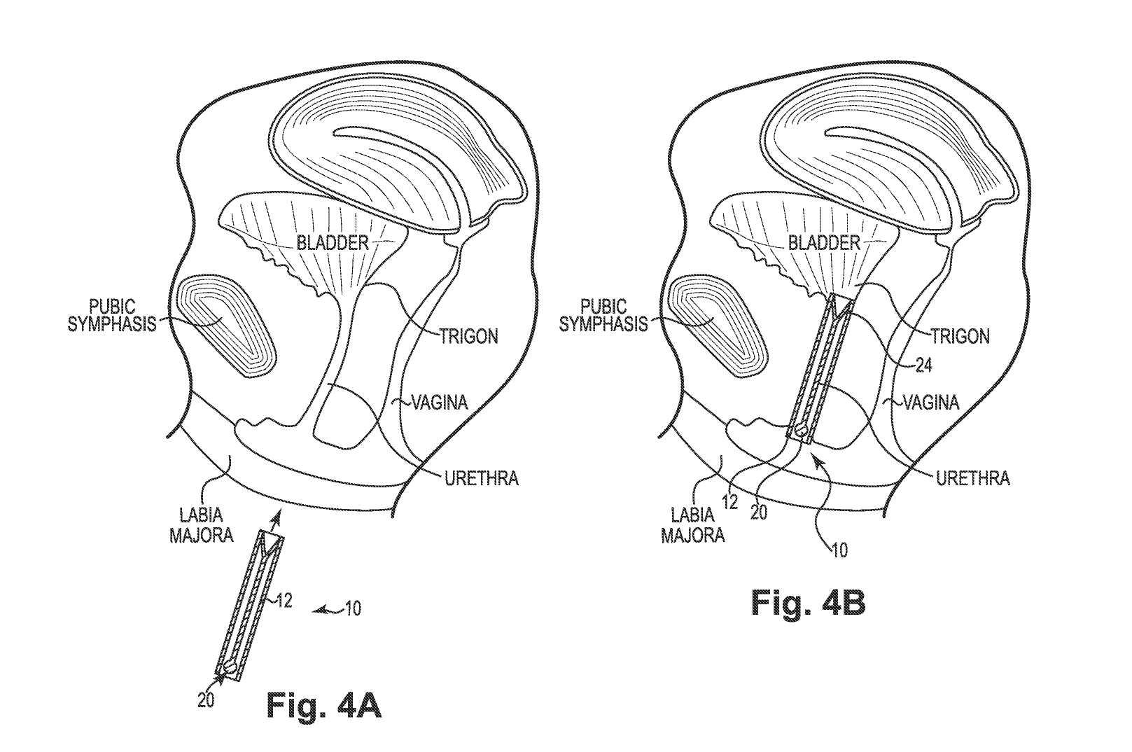

FIG. 2 is a side cross-sectional view and FIG. 3 is a top view of the device 20.

The device 20 is adapted to be inserted through the urethra with the proximal portion 24 inserted into the bladder while permitting the user to manually displace the proximal portion to allow urine to flow of the bladder. In one embodiment, the rod 22 is oriented along a longitudinal direction such as the longitudinal axis A, and the proximal portion 24 has a substantially triangular shape in longitudinal cross-section that is configured to block the neck to the urinary bladder and prevent urine from exiting the bladder until desired by the user. In one embodiment, the proximal portion 24 is substantially circular in lateral cross-section. The distal portion 26 is configured to be comfortable and identifiable by the user, and in one embodiment is provided in a spherical shape.

The rod 22 provides a connection between the distal portion 26 and the proximal portion 24 and is not provided as a urine conduit or other form of tubular urinary catheter. To this end, in one embodiment the rod 22 is a solid rod having sufficient column strength to rigidly attach the proximal portion 24 and the distal portion 26.

To accommodate insertion of the proximal portion 24 into the bladder through the urethra, in one embodiment the proximal portion 24 is fabricated from a compressible, flexible material that can be compacted for insertion into and removal from the bladder through the urethra.

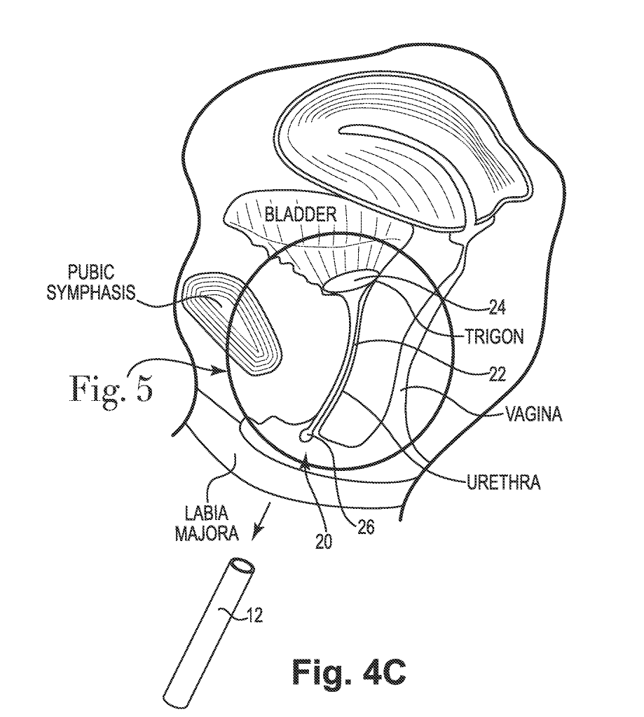

FIGS. 4A-4C are schematic views of the system 10 illustrated in FIG. 1 being placed in a female. The urethra extends from a distal location that forms an exit from the body to a proximal location connected with the bladder. The portion of the urethra that connects to the bladder is called a trigone, or that triangular region in cross-section that expands from the diameter of the urethra into the larger diameter of the bladder.

FIG. 4A is a schematic representation of the device 20 placed in the insertion tool 12 prior to delivery of the device 20 through the urethra into the bladder. The diameter of the insertion tool 12 is modeled after the diameter of cystoscopes, which are employed to visualize the interior of the urethra. The diameter of the insertion tool 12 is selected to be from 12 French to 20 French (or 4 mm to 6.7 mm), which configures the insertion tool 12 for passage through the urethra.

FIG. 4B illustrates one example of the system 10 initially inserted into a urethra of the user by a urologist. The urologist guides the insertion tool 12 through the urethra and ejects or otherwise pushes the device 20 out of the insertion tool 12 until the proximal portion 24 of the device 20 is engaged with the trigone region of the bladder.

FIG. 4C illustrates the insertion tool 12 removed from the urethra leaving the device 20 inserted into urethra with the proximal portion 24 engaged with the trigone of the bladder and the distal portion 26 located exterior and distal to the urethra.

One typical lifecycle envisioned for the system 10 is for a potential user to be evaluated in a clinic or other setting by a healthcare provider such as a uro-gynecologist; fitted for one of many available sizes of the device 20; tested/observed by the uro-gynecologist for continence with the device 20 in place; instructed in the use of the device 20; and discharged from the clinic, after which the user may remove the device 20 for cleaning or other reasons before self-replacing the device 20.

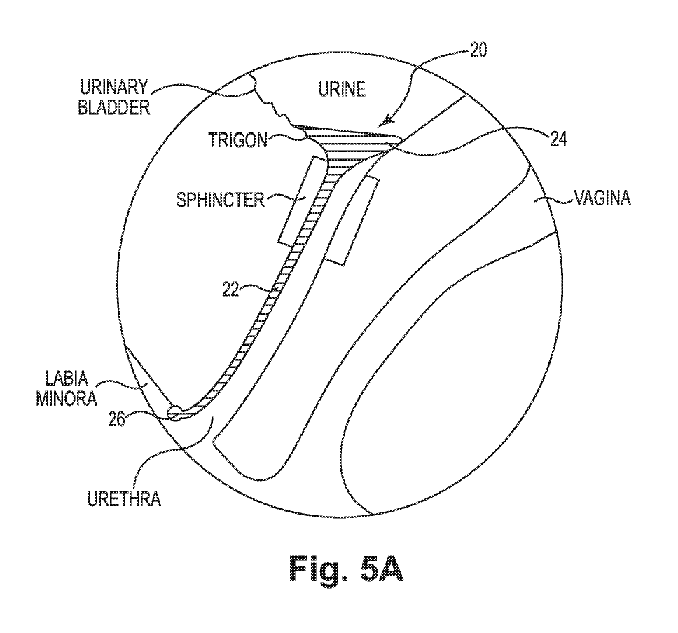

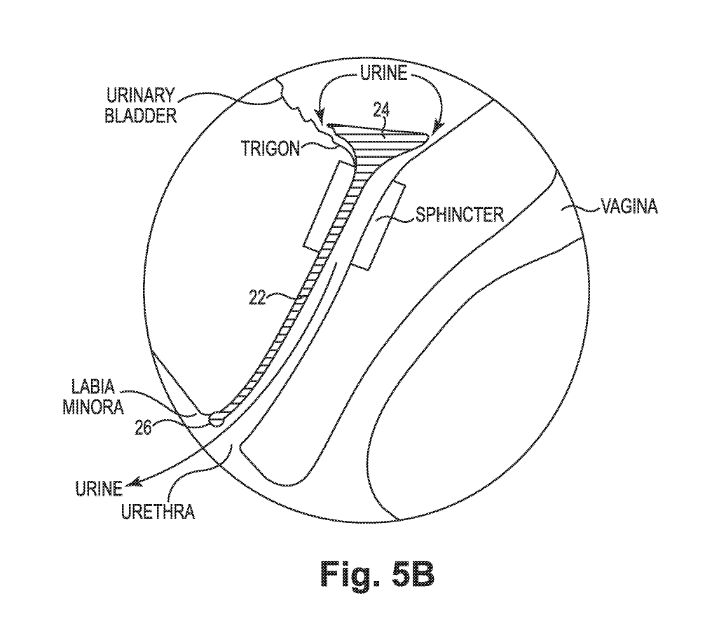

FIG. 5A is a side schematic view of the device 20 after insertion into the urethra. The proximal portion 24 of the device 20 is seated within the trigone region of the bladder and operates to occlude the exit of bladder. The rod extends from the proximal portion 24 to the distal portion 26. The distal portion 26 is located outside and distal to the urethra. In this position, the user has access to the distal portion 26 and is able to manipulate a position of the proximal 24 relative to the exit of the bladder.

In one embodiment, the proximal portion 24 is flexible and the lateral dimension W is selected to prevent the proximal portion from passing distal the exit of the urinary bladder in the trigone region; the lateral dimension D of the solid rod 22 is sized to allow urine to pass by the solid rod and exit the urethra; and a distal end of the distal portion 26 is sized to prevent the distal end from passing proximal to an exterior exit of the urethra.

FIG. 5B is a side schematic view of the device 20 manipulated to allow urine to pass from the bladder through the urethra. In the illustration, the proximal portion 24 has been displaced in a proximal direction to allow urine to escape the bladder through the urethra. The user voids urine by manually manipulating a location of the distal portion 24 in a proximal direction that lifts and separates the proximal portion 24 off of the seal formed at the trigone region of the bladder. In this position, the device 20 allows allow urine to flow around the proximal portion 24, alongside the rod 22, and out of the urethra. For example, the user may be instructed to provide an axial force against the distal portion 26 with a finger or other object. The force against the distal portion 26 moves the device 20 in a proximal direction and displaces the proximal portion 24 off of the trigone region of the bladder to form an opening for the escape of urine from the bladder. Removal of the force from the distal portion 26 of the device 20 operates to return the device to its initial position in which the proximal portion 24 blocks the exit of the bladder. The device 20 beneficially will return to its initial position under the force of gravity after the axial force applied to the distal portion 26 is removed. However, the accumulation of urine in the bladder also creates a force on the surface of the proximal portion 24 within the bladder to further assist in sealing the exit of the bladder.

The urinary sphincter is generally located at the upper one third proximal portion of the urethra. The urinary sphincter is a muscle that operates to contract against the urethra to impede the flow of urine out of the bladder. Generally, the tissue of the urethra is soft and pliable and has been described as having a nearly gelatinous consistency. Some people experience stress urinary incontinence or the sphincter muscle is deficient in fails to fully coapt the urethra to fully impede the flow of urine out of the bladder. The device 20 provides the beneficial advantage in that the rod 22 occupies some amount of space within the urethra that combines with the thickness of the wall of the urethra to allow the sphincter muscle to compress against the urethra and a portion of the rod, thus providing an improved seal in the area of the sphincter that impedes the flow of urine out of the bladder.

Embodiments of the device 20 provide a convenient and minimally invasive approach to providing urinary incontinence. The device 20 is configured to be biologically inert and functionally usable for months or years. With this in mind, in one embodiment the device 20 is fabricated as a single monolithic unit that is adapted for insertion through the urethra until the proximal portion 24 is inserted into the urinary bladder. In one embodiment, the device 20 is fabricated from plastic such as a thermoplastic elastomer, or polyethylene, or a silicone that is configured to allow the device 20 to move within the urethra and the bladder as the patient moves. That is, the device 20 is flexible and moves with the user.

The proximal portion 24 is desirably flexible and compressible to allow the proximal portion 24 to be inserted within the insertion tool 12 (FIG. 1A). The rod 22 is selected to have a sufficient column strength to allow an axial force against the distal portion 26 to transfer the force to the proximal portion 24. With this in mind, the rigidity or column strength of the rod 22 is preferably greater than the rigidity of the proximal portion 24.

In one embodiment, the device 20 is fabricated as a single integral or monolithic piece from a thermoplastic polymer in which the rod 22 has a first higher durometer than a durometer of the proximal portion 24. As an example, in one embodiment the durometer of the proximal portion 24 is less than approximately 25 on the durometer A scale and the durometer of the rod 22 is greater than approximately 25 on the durometer A scale. Exemplary values for the durometer of the device 20 include a durometer A scale of approximately 20 for the proximal portion 24 and a durometer A scale of approximately 40 for the durometer of the rod 22. In one embodiment, the durometer of the proximal portion 24 is low and configures the proximal portion 24 to be pliable enough to allow the user to remove the device 20 from her body by passing the proximal portion 24 out of the urinary bladder and out of the urethra.

In one embodiment, at least a portion of the device 20 is fabricated from metal such as stainless steel or a shape memory alloy such as the nickel-titanium alloy referred to as NiTiNOL. However, when the device 20 is formed of metal it can be expected to be less flexible and not move with the patient at the patient moves. Although fabrication of the device 20 from metal is acceptable from an engineering standpoint it may not be acceptable from a medical or end-user patient standpoint.

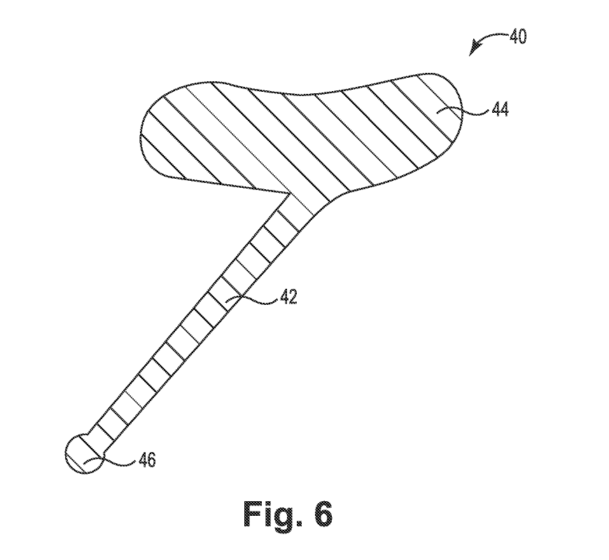

FIG. 6 is a side cross-sectional view of one embodiment of incontinence treatment device 40. The device 40 includes a rod 42 connected between a proximal portion 44 and a distal portion 46. In one embodiment, the shape of the proximal portion 44 in longitudinal cross-section is kidney shaped and so configured to block an exit of the bladder to the urethra and the trigone region. The rod 42 and the distal end 46 are configured in a manner similar to that described above for the rod 22 and the distal portion 26. It is desirable that the device 40 is fabricated from material that allows the proximal portion 44 to be flexible and compressible at least to the extent that allows the proximal portion 44 to be inserted through the urethra into the bladder.

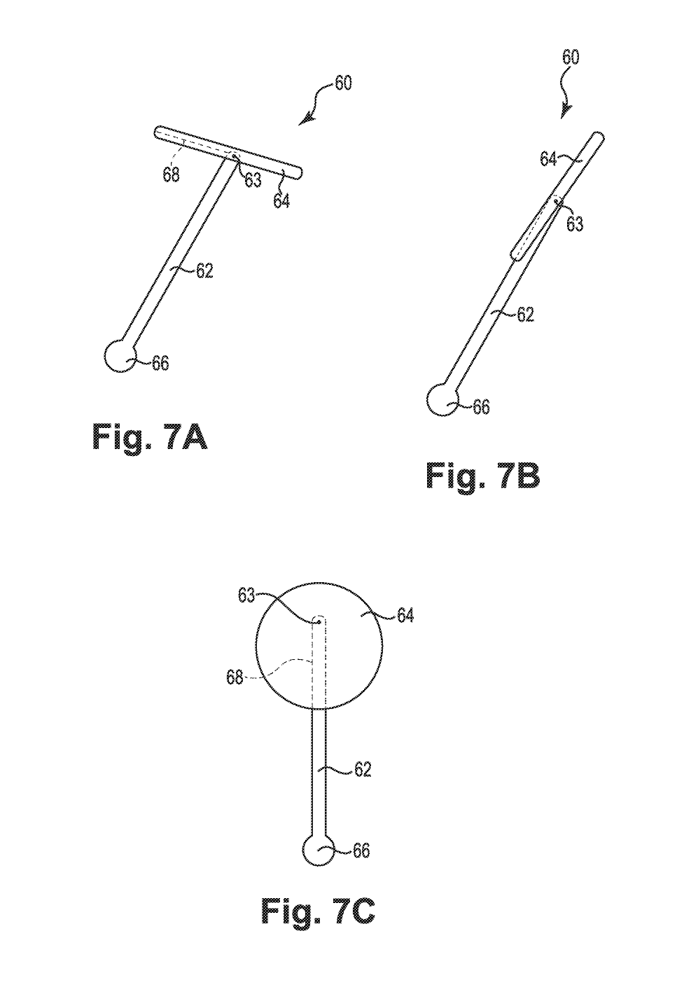

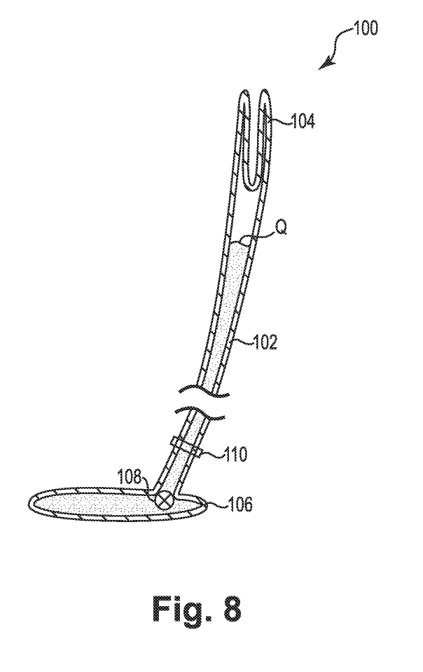

FIG. 7A-7C are side views of one embodiment of incontinence treatment device 60. Incontinence treatment device 60 includes a rod 62 connected by a pivot point 63 to a proximal portion 64, where the rod 62 terminates at a distal portion 66. In one embodiment, the proximal portion 64 includes a recess 68 that is sized to accommodate a section of the rod 62 when the proximal portion 64 is moved in to axial alignment with the rod 62 about pivot point 63. In one embodiment, the proximal portion 64 has a substantially rectangular shape in longitudinal cross-section and is substantially circular in lateral cross-section.

In one embodiment, the proximal portion 64 is configured to fold or otherwise align with the rod 62 to reduce a cross-sectional area of the device 60, which is useful when passing the device 60 through the urethra into the bladder.

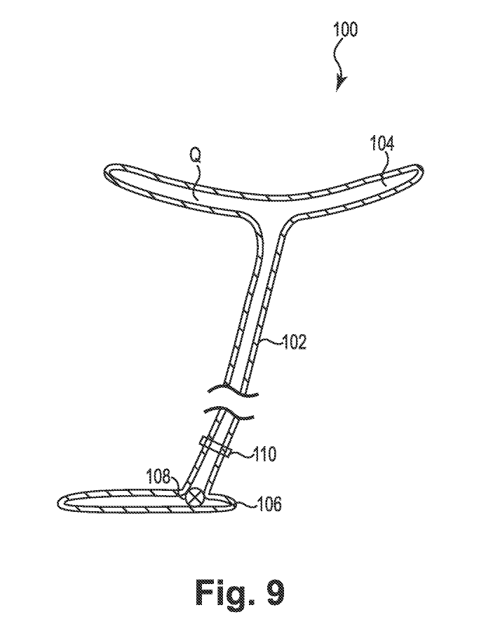

FIG. 8 is a side cross-sectional view of one embodiment of an incontinence treatment device 100. The device 100 includes a tube 102 extending between an inflatable proximal portion 104 and a pump 106. In general terms, a liquid Q is retained within the pump 106 and a portion of the tube 102. Compression of the pump 106 moves the fluid along the tube 102 into the inflatable proximal portion 104. In one embodiment, a check valve 108 is provided in the pump 106 to selectively maintain the liquid Q within the inflatable proximal portion 104. The check valve 108 is operable to allow the user to selectively move between a continent state in which the flow of urine from the bladder is blocked to a voiding state that allows the urine to the flow from the bladder through the urethra.

The tube 102 is preferably a kink resistant tube. Inflatable proximal portion 104 is configured to be collapsed when empty of liquid to assist in the placement through the urethra and the bladder. The proximal portion 104 is inflatable to provide a large area stopper that is configured to fit within the trigone region of the bladder to impede the flow of urine from the bladder through the urethra. The inflatable proximal portion 104 and the tube 102 are both flexible and so configured to move as the user moves. Suitable material for fabricating the inflatable proximal portion 104 and the tube 102 include plastic material, such as silicone or polyethylene or thermoplastic elastomers.

The pump 106 is sized for implantation, for example, subcutaneously within the labia majora of the user. It may become desirable to eventually replace the tube 102 or the inflatable proximal portion 104, and with this in mind one embodiment provides a connector 110 that allows the tube 102 and the proximal portion 104 to be replaced and reconnected with the pump 106.

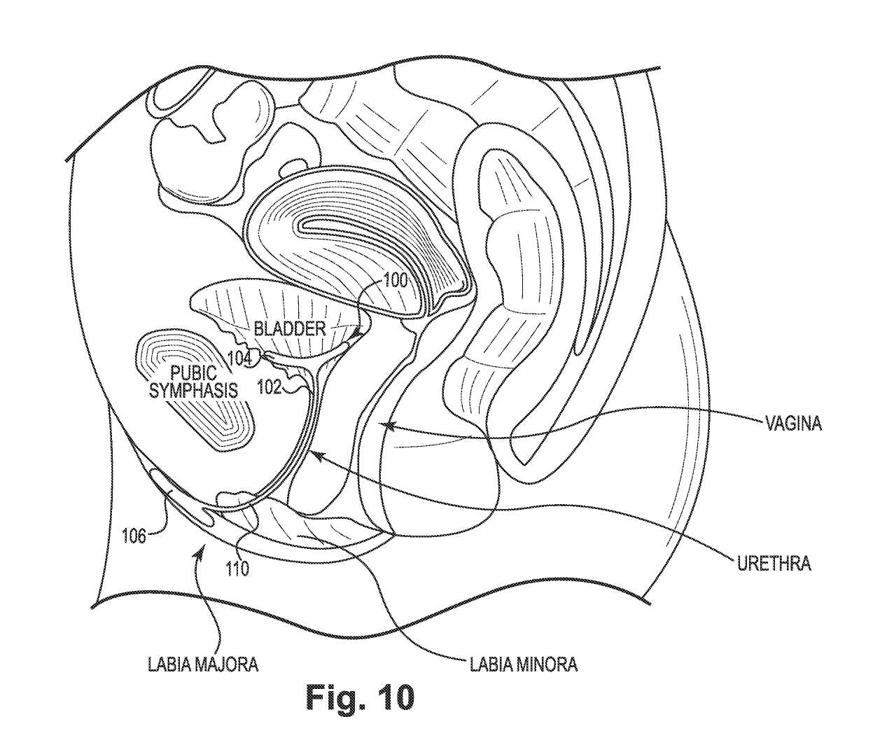

FIG. 9 is a side cross-sectional view of the device 100. The pump 106 has been compressed to drive the liquid Q from the pump 106 and the tube 102 into the proximal portion 104. The proximal portion 104 has become inflated and the liquid Q is prevented from flowing back into the pump 106 by the check valve 108. In this configuration, the inflated proximal portion 104 provides a substantially greater lateral area than the tube 102, and thus functions as a seal to prevent the flow of urine from the bladder through the urethra.

In one embodiment, the device 100 is a closed system containing the liquid Q and the pump is configured to move the liquid Q out of the tube 102 into the inflatable proximal portion 104. For example, in one embodiment the check valve 108 is configured to retain the liquid Q in the inflatable proximal portion 104 after operation of the pump 106 to provide the user with a state of continence. The proximal portion 104 will cover the trigone region of the bladder and impede the exit of urine from the bladder after inflation of the proximal portion 104. In one embodiment, the check valve 108 is conveniently located within the pump 106 to allow the user to selectively displace a valve off of a valve seat to allow the liquid Q to drain from the proximal portion 104 back into the pump 106. With this in mind, the check valve 108 is suitably provided as a ball valve that is biased by a spring or other such suitable valve arrangements.

FIG. 10 is a schematic view of the device 100 placed within a female user. In one embodiment, the pump 106 is subcutaneously implanted into the labia majora and the tube 102 extends through the urethra. The proximal portion 104 is placed within the bladder and is inflated to provide the user with a continent state and is deflated to allow the user to void urine. In this manner, the user has access to the distal portion (the pump 106), which facilitates inflating and deflating the proximal portion 104.

In one embodiment, the tube 102 is inserted into the urethra and a lateral dimension of the tube is less than approximately 4 mm and so configured to allow urine to pass an exterior of the tube 102 and exit the urethra.

In one embodiment, the inflatable proximal portion 104 is insertable through the urethra into the urinary bladder and the pump 106 is placed alongside or even attached to the labia majora, for example with adhesive. When inflated, the inflatable proximal portion 104 is substantially circular in lateral cross-section and so configured to block the exit of the urinary bladder to the urethra.

Embodiments provide for placement of the device 100 in a male user in which the proximal portion 104 is placed within the bladder and the pump 106 is implanted in the scrotum. The pump 106 is operable to inflate the proximal portion 104 to prevent urine from exiting the bladder and is also operable to deflate the proximal portion 104 to allow urine to exit the bladder alongside the tube 102.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of medical devices as discussed herein. Therefore, it is intended that this invention be limited only by the claims and the equivalents thereof.

What is claimed is:

1. An incontinence treatment device comprising:
a proximal portion that is insertable into a urinary bladder;
a solid rod connected to the proximal portion, the solid rod configured for placement in an urethra; and
a distal portion connected to the solid rod, the solid rod having a length that adapts the distal portion to be positioned outside and distal to the urethra with the proximal portion positioned in the urinary bladder;
wherein the proximal portion has a lateral dimension that is at least a factor of 3 greater than a lateral dimension of the solid rod and is so configured to block an exit of the urinary bladder and impede flow of urine out of the urinary bladder; and
wherein an axial force applied to the distal portion displaces the proximal portion away from the exit of the urinary bladder to allow urine to pass through the urethra.

2. The device of claim 1, wherein the solid rod is oriented along a longitudinal direction extending between the proximal portion and the distal portion, and the proximal portion has a substantially triangular shape in longitudinal cross-section.

3. The device of claim 1, wherein the solid rod is oriented along a longitudinal direction extending between the proximal portion and the distal portion, and the proximal portion has a substantially kidney shape in longitudinal cross-section.

4. The device of claim 1, wherein the solid rod is oriented along a longitudinal direction extending between the proximal portion and the distal portion, and the proximal portion has a substantially rectangular shape in longitudinal cross-section.

5. The device of claim 4, wherein the proximal portion is substantially circular in lateral cross-section.

6. The device of claim 1, wherein proximal portion is compressible and adapted to be packed into a tube that is sized for delivery through the urethra to the urinary bladder.

7. The device of claim 1, wherein the solid rod has a durometer that is harder than a durometer of the proximal portion.

8. The device of claim 1, wherein the lateral dimension of the solid rod is less than approximately 4 mm and so configured to allow urine to pass the solid rod and exit the urethra.

9. The device of claim 1, wherein a distal end of the distal portion is spherical.

10. The device of claim 1, wherein:
the proximal portion is flexible and the lateral dimension is selected to prevent the proximal portion from passing distal the exit of the urinary bladder;
the lateral dimension of the solid rod is sized to allow urine to pass by the solid rod and exit the urethra; and
a distal end of the distal portion is sized to prevent the distal end from passing proximal to an exterior exit of the urethra.

11. The device of claim 1, wherein the solid rod is connected to the proximal portion by a pivot attachment configured to allow the proximal portion to move relative to the solid rod.

12. The device of claim 1, wherein the proximal portion and the solid rod and the distal portion are formed as a single monolithic body.

13. The device of claim 12, wherein the single monolithic body is formed from plastic.

14. The device of claim 13, wherein the proximal portion has a first durometer that is lower than a durometer of the solid rod.

15. An incontinence treatment system comprising:
an insertion tool; and
a device insertable into and ejectable from the insertion tool, the device comprising:
a proximal portion that is insertable into a urinary bladder;
a solid rod connected to the proximal portion, the solid rod configured for placement in an urethra; and
a distal portion connected to the solid rod, the solid rod having a length that adapts the distal portion to be positioned outside and distal to the urethra with the proximal portion positioned in the urinary bladder;
wherein the proximal portion has a lateral dimension that is greater than a lateral dimension of the solid rod and is so configured to block an exit of the urinary bladder and impede flow of urine out of the urinary bladder, and the distal portion is movable in a proximal direction to move the proximal portion away from the exit of the urinary bladder to allow urine to pass through the urethra.

16. The system of claim 15, wherein the proximal portion, the solid rod, and the distal portion are formed as a single monolithic piece.

17. The system of claim 16, wherein the proximal portion has a first durometer that is lower than a durometer of the solid rod.

18. The system of claim 17, wherein the first durometer of the proximal portion allows a user to remove the device from her body by passing the proximal portion out of the urinary bladder and out of the urethra.

19. The system of claim 15, wherein the lateral dimension of the solid rod is less than approximately 4 mm and so configured to allow urine to pass the solid rod and exit the urethra.

20. The system of claim 15, wherein the proximal portion is substantially circular in lateral cross-section.
